(19) United States
(12) Patent Application Publication
(10) Pub. No.: US 2025/0090015 A1
(43) Pub. Date: Mar. 20, 2025

(54) POLARIZATION SENSITIVE OPTICAL COHERENCE TOMOGRAPHY FOR VISUALIZATION OF VITREOUS OPACITIES

(71) Applicant: Alcon Inc., Fribourg (CH)

(72) Inventors: Sahar Hosseinzadeh Kassani, Lake Forest, CA (US); Parisa Rabbani, Aliso Viejo, CA (US); Marcia Ong, Fort Worth, TX (US); Lingxiao Yang, Urbana, IL (US)

(73) Assignee: Alcon Inc., Fribourg (CH)

(21) Appl. No.: 18/828,214

(22) Filed: Sep. 9, 2024

Related U.S. Application Data
(60) Provisional application No. 63/582,923, filed on Sep. 15, 2023.

(51) Int. Cl. A61B 3/10; G02B 27/28; G16H 20/40; G16H 40/63

(52) U.S. Cl. CPC A61B 3/102; G16H 20/40; G16H 40/63; G02B 27/283

(57) ABSTRACT

A system of visualizing a target site in an eye, using a polarization sensitive optical coherence tomography (PS-OCT) device, includes a controller having at least one processor and at least one non-transitory, tangible memory on which instructions are recorded. The target site is one or more vitreous opacities in the vitreous humor of the eye. The controller is configured to receive PS-OCT data and determine at least one parameter corresponding to birefringence properties of collagen fibrils in the vitreous humor based on the PS-OCT data. The at least one parameter includes respective spacing of the collagen fibrils. The controller is configured to determine a respective location of the one or more vitreous opacities when the at least one parameter is outside a predefined range and generate a control signal adapted for guiding a treatment beam at the respective location of the one or more vitreous opacities.

### CROSS REFERENCE TO RELATED APPLICATION

The present application claims the benefit of priority to U.S. Provisional Application No. 63/582,923 filed Sep. 15, 2023, which is hereby incorporated by reference in its entirety.

### INTRODUCTION

The disclosure relates to a system and method for visualizing vitreous opacities in an eye using polarization sensitive optical coherence tomography (“PS-OCT”). A common condition affecting vision quality is the presence of vitreous opacities, sometimes referred to as floaters, in the vitreous humor of the eye. Vitreous opacities may appear as spots or shadows of various shapes that appear to float in the field of vision of the patient, and scatter light entering the eye. The origin of the vitreous opacities may be microscopic collagen fibers within the vitreous humor. Treatment of the vitreous opacity may include vitrectomy or laser vitreolysis. Because the vitreous cavity and the retina are deeper than anterior tissues such as the cornea and the lens, effective visualization and delivery of treatment for vitreous opacities is often challenging.

### SUMMARY

Disclosed herein is a system and method system of visualizing a target site in an eye, using a polarization sensitive optical coherence tomography (“PS-OCT” hereinafter) device. The system includes a controller having a processor and a tangible, non-transitory memory on which instructions are recorded. The target site is one or more vitreous opacities (“one or more” omitted henceforth) in the vitreous humor of the eye. The PS-OCT device includes a source adapted to generate a PS-OCT source beam and a polarizer adapted to control a polarization of the PS-OCT source beam. The PS-OCT device includes one or more polarization sensitive detectors adapted to detect an interference pattern based in part on a reflected PS-OCT beam and generate PS-OCT data relating to the interference pattern.

The controller is configured to receive the PS-OCT data and determine at least one parameter (“at least one” omitted henceforth) corresponding to birefringence properties of collagen fibrils in the vitreous humor based on the PS-OCT data. The parameter includes the respective spacing of the collagen fibrils. The controller is configured to determine a respective location of the vitreous opacities when the parameter is outside a predefined range and generate a control signal adapted for guiding a treatment beam at the respective location of the vitreous opacities.

The parameter may include a respective orientation of the collagen fibrils. The PS-OCT device may include a beam splitter adapted to split the PS-OCT source beam into a sample beam propagating in a sample arm and a reference beam propagating in a reference arm, the reference arm having a reference mirror. A polarizing beam splitter is adapted to split the reflected PS-OCT beam into two orthogonally polarized components. The reflected PS-OCT beam is a combination of respective reflected beams of the sample beam and the reference beam.

The PS-OCT device may include a first quarter-wave plate adapted to convert the sample beam into a polarized sample beam incident upon the target site, and a second quarter-wave plate adapted to convert the reference beam into a polarized reference beam incident upon the reference mirror. The two orthogonally polarized components include a vertically polarized component and a horizontally polarized component. The polarization sensitive detectors may include a vertical detector adapted to receive the vertically polarized component, and a horizontal detector adapted to receive the horizontally polarized component. In some embodiments, the first quarter-wave plate is oriented at an angle of 22.5 degrees and the second quarter-wave plate is oriented at the angle of 45 degrees.

The PS-OCT device may include a first channel and a second channel adapted to respectively detect a signal from the PS-OCT data in a first orthogonal polarization state and a second orthogonal polarization state. The signal is converted to a fast-Fourier transformed signal. A phase retardation mode may be adapted to display the PS-OCT data. The phase retardation mode is based on a retardation factor (δ), represented as

$$[ δ = {tan}^{- 1} ( F * \frac{{A}_{2}}{{A}_{1}} ) ] ,$$

where F is a calibration factor, and A₁, A₂ are respective amplitudes of the fast-Fourier transformed signal from the first channel and the second channel.

An optical axis mode may be adapted to display the PS-OCT data. The optical axis mode is based on an optical factor (θ), represented as

$$[ θ = \frac{π}{2} - \frac{( {ϕ}_{1} - {ϕ}_{2} )}{2} ] ,$$

where ϕ₁ and ϕ₂ are respective phases of the fast-Fourier transformed signal from the first channel and the second channel.

In some embodiments, a laser unit is adapted to selectively generate the treatment beam directed towards the one or more vitreous opacities, the treatment beam including a plurality of ultra-short laser pulses. The plurality of ultra-short laser pulses may define a respective time duration of between about a femtosecond and about 50 picoseconds. The laser unit and the PS-OCT device may have a shared aperture for guiding the treatment beam and the PS-OCT beam towards the target site, the shared aperture being centered about a center axis. The treatment beam may travel at an off-axis angle from the center axis, the off-axis angle being at or above 15 degrees.

Disclosed herein is a method of visualizing a target site in an eye using a polarization sensitive optical coherence tomography (PS-OCT) device in a system having a controller with at least one processor and at least one non-transitory, tangible memory. The method includes generating a PS-OCT source beam, via a light source in the PS-OCT device, and controlling a polarization of the PS-OCT source beam, via a polarizer in the PS-OCT device. The target site is one or more vitreous opacities in a vitreous humor of the eye. The method includes detecting an interference pattern based in part on a reflected PS-OCT beam and generating PS-OCT data relating to the interference pattern, via one or more polarization sensitive detectors in the PS-OCT device.

The method includes receiving the PS-OCT data, via the controller, and determining at least one parameter corresponding to birefringence properties of collagen fibrils in the vitreous humor based on the PS-OCT data, via the controller. The parameter includes a respective spacing of the collagen fibrils. The method includes determining a respective location of the one or more vitreous opacities when the at least one parameter is outside a predefined range, via the controller. The method includes generating a control signal adapted for guiding a treatment beam at the respective location of the one or more vitreous opacities, via the controller.

The above features and advantages and other features and advantages of the present disclosure are readily apparent from the following detailed description of the best modes for carrying out the disclosure when taken in connection with the accompanying drawings.

### BRIEF DESCRIPTION OF THE DRAWINGS

FIG. 1 is a schematic illustration of a system for visualizing an eye using a PS-OCT device, the system having a controller;

FIG. 2 is a schematic diagram illustrating an eye with vitreous opacities;

FIG. 3 is a schematic diagram illustrating the birefringence effect on polarized light;

FIG. 4 is a schematic flowchart for a method executable by the controller of FIG. 1;

FIG. 5 is a schematic magnified view of example healthy collagen fibrils in the vitreous humor of an eye;

FIG. 6 is a schematic magnified view of example unhealthy collagen fibrils in the vitreous humor;

FIG. 7 is a schematic diagram of an example architecture for applying portions of the method of FIG. 4;

FIG. 8 is a schematic view of an example image generated with PS-OCT data, in a phase retardation mode; and

FIG. 9 is a schematic view of an example image generated with PS-OCT data, in an optical axis mode.

Representative embodiments of this disclosure are shown by way of non-limiting example in the drawings and are described in additional detail below. It should be understood, however, that the novel aspects of this disclosure are not limited to the particular forms illustrated in the above-enumerated drawings. Rather, the disclosure is to cover modifications, equivalents, combinations, sub-combinations, permutations, groupings, and alternatives falling within the scope of this disclosure as encompassed, for instance, by the appended claims.

### DETAILED DESCRIPTION

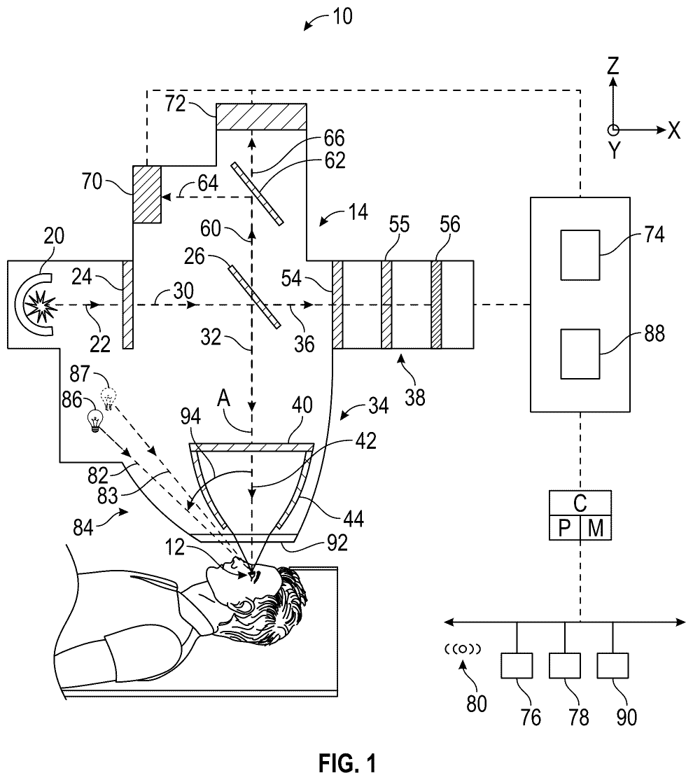

Referring to the drawings, wherein like reference numbers refer to like components, FIG. 1 schematically illustrates a system 10 for visualizing a target site in an eye 12 with data captured via a polarization sensitive optical coherence tomography device 14. Optical coherence tomography is a noninvasive imaging technology using low-coherence interferometry to generate high-resolution images of ocular structure. Images generated by conventional optical coherence tomography systems are intensity-based and useful for many purposes, such as identification and assessment of ocular diseases. However, they lack tissue-specific contrast. In other words, there is little differentiation between different types of tissues. This makes interpretation of images challenging.

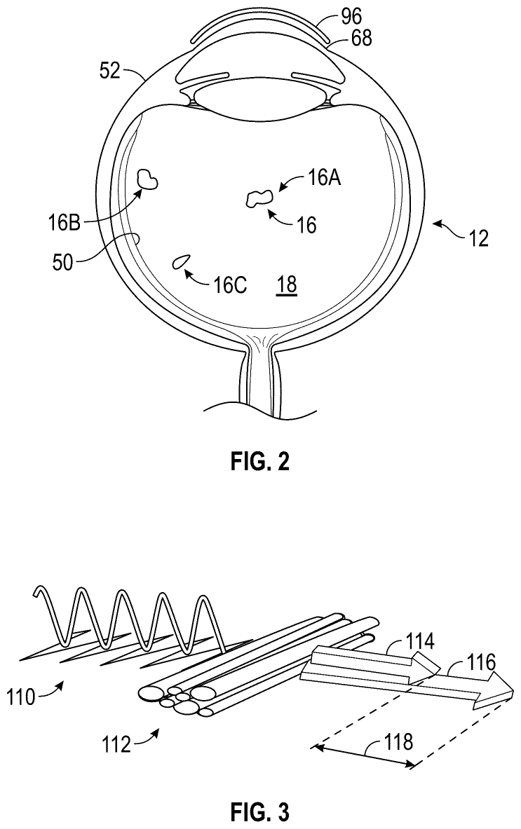

As described below, the system 10 is configured to identify and treat one or more vitreous opacities 16 (see FIG. 2) in the eye 12 using polarization sensitive optical coherence tomography (“PS-OCT” hereinafter). Referring to FIG. 2, the vitreous opacities 16 may be located at various positions in the vitreous humor 18 of the eye 12, as indicated by first vitreous opacity 16A, second vitreous opacity 16B, and third vitreous opacity 16C (see FIG. 2). The vitreous humor includes 18 approximately 98% water, hyaluronic acid, proteoglycans and collagen fibers. The collagen forms uniform and parallel fibrils that increase in density with age.

The main component of the vitreous opacities 16 is collagen. Tissues containing collagen have birefringence properties, which is an optical property of a material whose refractive index is dependent upon the polarization orientation and direction of propagation of light. As shown in FIG. 3, when polarized light 110 passes through a material 112 with birefringence properties, such as tissue with collagen fibers, a first polarization orientation 114 of the light slows down with respect to the second polarization orientation 116, resulting in a phase delay 118. The magnitude of the phase delay 118 may be used to characterize the structure of the material 112.

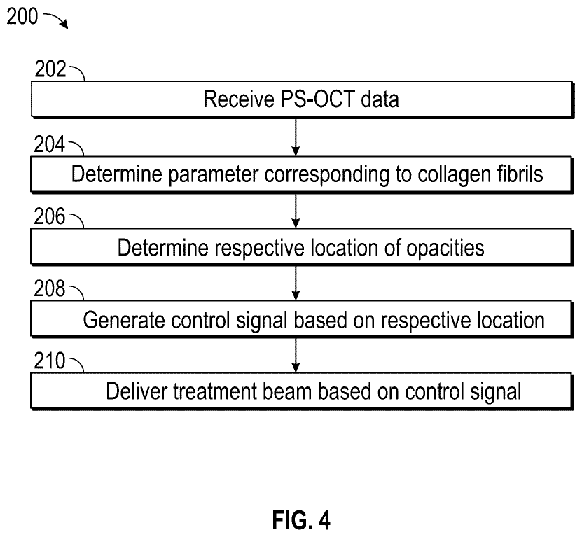

Referring to FIG. 1, the system 10 includes a controller C having at least one processor P and at least one memory M (or non-transitory, tangible computer readable storage medium) on which instructions are recorded for executing method 200 for visualizing vitreous opacities 16 in the eye 12 using the PS-OCT device 14. Method 200 is shown in and described below with reference to FIG. 4.

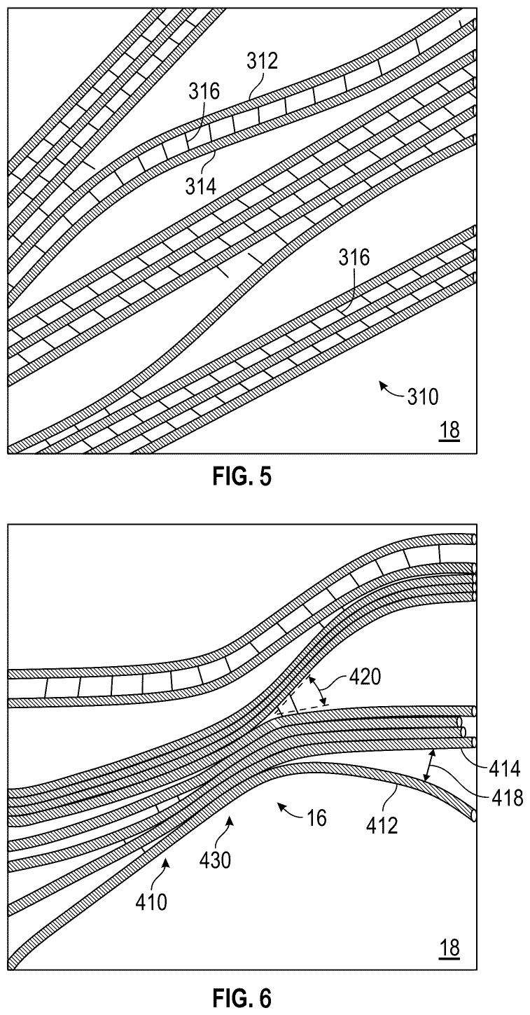

The system 10 enables identification of tissue-specific contrasts which is used to characterize the vitreous opacities 16. Referring now to FIG. 5, an example view of relatively healthy collagen fibrils 310 in the vitreous humor 18 is shown. Referring to FIG. 5, the collagen fibrils 310 form an extended network by being organized into bundles (e.g., first bundle 312 and second bundle 314) that are relatively aligned or parallel. The collagen fibrils 310 are both linked together and spaced apart by chondroitin sulfate chains 316. In a relatively healthy eye, the chondroitin sulfate chains 316 may be substantially orthogonal to the first bundle 312 and second bundle 314.

FIG. 6 is a schematic example view of relatively unhealthy collagen fibrils 410 in the vitreous humor 18. The location of vitreous opacities 16 may be characterized by the presence of aggregated, cross-linked collagen fibrils 410. As shown in FIG. 6, the collagen fibrils 410 are clumped together at various points with the bundles of collagen fibrils 410, such as first bundle 412 and second bundle 414, being relatively unaligned. Various factors cause the collagen fibrils 410 to clump together, including the loss of certain types (e.g., type IX) of collagen from the fibril surface. The factors may include age or various eye conditions such as posterior vitreous detachment, myopic vitreopathy etc.

Referring now to FIG. 1, the system 10 includes a light source 20 adapted to generate a PS-OCT source beam 22. The light source 20 may be a swept-source laser or other suitable OCT source available to those skilled in the art. It is to be understood that the PS-OCT device 14 may take many different forms and include multiple and/or alternate components.

As shown in FIG. 1, the PS-OCT source beam 22 is propagated through a polarizer 24 to a beam splitter 26. The polarizer 24 receives the incident PS-OCT source beam and transmits a polarized PS-OCT source beam 30 with a known polarization. At the beam splitter 26, the polarized PS-OCT source beam 30 is split into two components: a sample beam 32 that is propagated through a sample arm 34, and a reference beam 36 that is propagated through a reference arm 38. The beam splitter 26 is a non-polarizing 50/50 beam splitter.

Referring to FIG. 1, in the sample arm 34, the sample beam 32 passes through a first quarter-wave plate 40 which converts the sample beam 32 into polarized light with a known polarization state such that a polarized sample beam 42 is incident upon the eye 12. Here the first quarter-wave plate 40 may be angled at 45 degrees. A collimation unit 44 may be employed to guide the polarized sample beam 42. Referring to FIG. 2, the incident beam (polarized sample beam 42 in FIG. 1) is reflected from the retina 50 and/or sclera 52 of the eye 12 and is diffracted, reflected, and/or refracted by the vitreous opacities 16 in the vitreous humor 18. The reflected sample beam then travels back through to the PS-OCT device 14, e.g., through various combinations of optical devices (not shown).

On the reference arm 38, the reference beam 36 passes through a second quarter-wave plate 54, and a reference mirror 56. The second quarter-wave plate 54 converts the polarization state of the returning reference beam into a polarization state with two equal orthogonal polarization components. Here the second quarter-wave plate 54 may be angled at 22.5 degrees. The reference arm 38 may include a dispersion compensator 55 positioned between the quarter-wave plate 54 and the reference mirror 56, for controlling the chromatic dispersion of the beam.

Referring to FIG. 1, when the reflected sample beam (reflected from portions of the eye 12) and the reflected reference beam (reflected from the reference mirror 56) are combined at the beam splitter 26, they form a reflected PS-OCT beam 60, which is directed to a polarizing beam splitter 62. The reflected PS-OCT beam 60 is a combination of respective reflected beams of the sample beam and the reference beam. At the polarizing beam splitter 62, the reflected PS-OCT beam 60 is split into two orthogonally polarized components, for example, a vertically polarized component 64 and a horizontally polarized component 66. The term “horizontal” refers to X and Y-directions in the X-Y plane, which may be defined as a plane roughly perpendicular to an apex of the cornea 68. The term “vertical” may refer to the Z-direction in the Z-plane, defined as a plane roughly perpendicular to the X-Y plane. It is understood that the origin and orientation of the XYZ coordinate system may be varied based on the application.

Referring to FIG. 1, the system 10 includes one or more polarization sensitive detectors, such as vertical polarization sensitive detector 70 and horizontal polarization sensitive detector 72, adapted to receive the reflected PS-OCT beam 60. In the example shown in FIG. 1, the vertical polarization sensitive detector 70 is adapted to receive the vertically polarized component 64 and the horizontal polarization sensitive detector 72 is adapted to receive the horizontally polarized component 66. The polarization sensitive detectors detect an interference pattern of the reflected PS-OCT beam 60 and generate data relating to the interference pattern. This data may be transmitted to the controller C, via an OCT processor 74.

Referring to FIG. 1, the controller C may be configured to process signals from the PS-OCT device 14 for broadcasting on a display 76. The display 76 may include, but is not limited to, a high-definition or ultra-high-definition television, smart-eyewear, projectors, one or more computer screens, laptop computers, tablet computers and may include a touchscreen. Referring to FIG. 1, the controller C may be configured to receive and transmit data through a user interface 78. The user interface 78 may be installed on a smartphone, laptop, tablet, desktop or other electronic device.

The various components of the system 10 of FIG. 1 may communicate via a network 80. The network 80 may be a bus implemented in various ways, such as for example, a serial communication bus in the form of a local area network. The local area network may include, but is not limited to, a Controller Area Network (CAN), a Controller Area Network with Flexible Data Rate (CAN-FD), Ethernet, blue tooth, WIFI and other forms of data connection. The network 80 may be a Wireless Local Area Network (LAN) which links multiple devices using a wireless distribution method, a Wireless Metropolitan Area Networks (MAN) which connects several wireless LANs or a Wireless Wide Area Network (WAN). Other types of connections may be employed.

Referring now to FIG. 4, a flow chart of method 200 executable by the controller C of FIG. 1 is shown. Method 200 need not be applied in the specific order recited herein and some blocks may be omitted. The memory M can store controller-executable instruction sets, and the processor P can execute the controller-executable instruction sets stored in the memory M.

Beginning at block 202 of FIG. 4, the controller C is configured to receive the PS-OCT data captured by the PS-OCT device 14. Advancing to block 204 of FIG. 4, the controller C is configured to analyze the PS-OCT data to determine at least one parameter corresponding to birefringence properties of collagen fibrils in the vitreous humor. The parameter includes a respective spacing of the collagen fibrils 410, such as the respective spacing 418 between the first bundle 412 and the second bundle 414 shown in FIG. 6. The parameter may include respective orientation 420 of the collagen fibrils 410, e.g., whether the bundles are parallel or becoming flared. The respective orientation 420 may be represented by an angle between respective bundles.

Proceeding to block 206 of FIG. 4, the controller C may be configured to determine a respective location (such as location 430 in FIG. 6) of the vitreous opacities 16 when the parameter is outside a predefined range of values, e.g., the respective spacing 418 being below a threshold value. For example, the respective spacing 418 between the first bundle 412 and the second bundle 414 is about zero at the location 430, which may be designated as a vitreous opacity 16. In some embodiments, the controller C may generate a multi-factor score based on the PS-OCT data for each grid or segment of a spatial image of the vitreous humor 18.

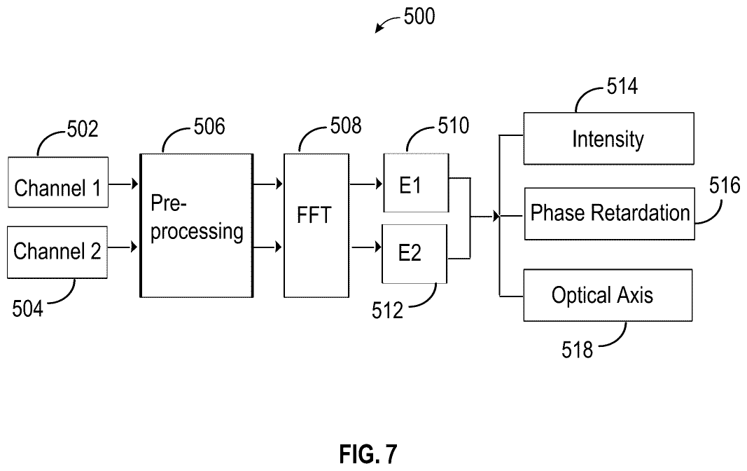

Referring to FIG. 7, a workflow diagram for processing the PS-OCT data is shown. The PS-OCT device 14 of FIG. 1 may include a first channel 502 and a second channel 504 (shown in FIG. 7), adapted to detect respective signals from the PS-OCT data in two orthogonal polarization states: a first orthogonal polarization state and a second orthogonal polarization state (e.g., vertical and horizontal polarized states). Referring to FIG. 7, raw data from the first channel 502 and the second channel 594 is transmitted to a pre-processing unit 506, where a plurality of pre-processing steps are carried. These may include padding, windowing, background reduction, filtering etc.

The respective signals output by the pre-processing module 506 undergo a fast Fourier transformation, as indicated by block 508 in FIG. 7. After the Fourier-transformation, the respective signals from the first channel 502 and the second channel 504 are each processed to obtain an OCT signal, E₁ and E₂, respectively, as shown in blocks 510 and 512. The fast-Fourier transformed signal from the first channel 502 may be represented as E₁=A₁e^{iϕ1}, where A₁ and ϕ₁ denote an amplitude and a phase, respectively. The fast-Fourier transformed signal from the second channel 504 may be represented as E₂=A₂e^{iϕ2}, where A₂ and ϕ₂ denote an amplitude and a phase, respectively. Referring to FIG. 7, the signals from the PS-OCT data may be displayed using an intensity mode 514, a phase retardation mode 516, and an optical axis mode 518.

The phase retardation mode 516 may be based on a retardation factor (δ), represented as

$$[ δ = {tan}^{- 1} ( F * \frac{{A}_{2}}{{A}_{1}} ) ] .$$

Here F is a calibration factor, and A₁, A₂ are respective amplitudes of the fast-Fourier transformed signal from the first channel 502 and the second channel 504. Phase retardation is related to the tissue birefringence (An) as:

$$δ = \frac{2 π L}{λ} Δ n ,$$

where λ is the wavelength of light, and L is the imaging depth. In one embodiment, F is a factor that is calibrated from surface reflection data to account for imperfect circular polarization in the sample arm 34. As noted above, referring to FIG. 1, the sample arm 34 may be converted to a circularly polarized state by adjusting the polarization controller in the beam path and inserting a quarter wave plate 40.

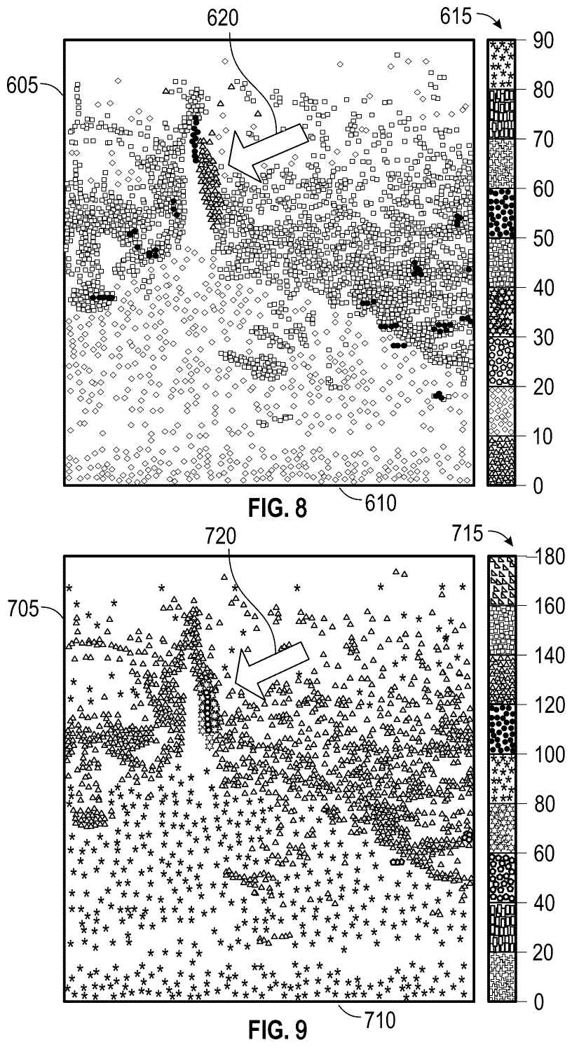

FIG. 8 is a schematic view of an example PS-OCT image displayed in the phase retardation mode 516. The arrow 620 highlights contrast due to collagen content (e.g., in the vitreous humor 18). The phase retardation mode 516 is visualized in a range from 0 to 90 degrees. The data is divided into individual blocks of 10 degrees each, as indicated by legend 615. The vertical axis 605 and horizontal axis 610 denote spatial dimensions.

The optical axis mode 518 may be based on an optical factor (θ), represented as

$$[ θ = \frac{π}{2} - \frac{( {ϕ}_{1} - {ϕ}_{2} )}{2} ] ,$$

where ϕ₁ and ϕ₂ are respective phases of the fast-Fourier transformed signal from the first channel 502 and the second channel 504. FIG. 9 is a schematic view of an example PS-OCT image displayed in the optical axis mode 518. The arrow 720 highlights contrast due to collagen content (e.g., in the vitreous humor 18). The optical axis mode 516 is visualized in a range from 0 to 180 degrees. The data is divided into individual blocks of 20 degrees each, as indicated by legend 715. The vertical axis 705 and horizontal axis 710 denote spatial dimensions.

The intensity mode 514 may be based on an intensity factor (I), represented as [I=20 log \sqrt{(A₁²+A₂²)}]. Here A₁, A₂ are respective amplitudes of the fast-Fourier transformed signal from the first channel 502 and the second channel 504.

Per block 208 of FIG. 4, the method 200 includes generating a control signal for guiding at least one treatment beam 82 at the respective location (such as location 430 in FIG. 6) of the vitreous opacities 16. Per block 210, a treatment beam 82 (see FIG. 1) may be delivered based on this control signal. Referring back to FIG. 1, the system 10 may include a laser unit 84 configured to selectively generate at least one treatment beam 82 directed towards the vitreous opacities 16, via a laser source 86. The treatment beam 82 may include a plurality of ultra-short laser pulses, each having a time duration of between about a femtosecond (10⁻¹⁵ seconds) and about 50 picoseconds (50×10⁻¹² seconds). Data from the control signal may be processed through a laser processor 88 and used to optimize the treatment beam 82 in order to at least partially vaporize or disintegrate the vitreous opacities 16.

The laser source 86 may be a femtosecond laser or a picosecond laser and may emit light with a wavelength of about 1,050 nm. In one example, the laser source 86 is configured to deliver infrared radiation at a wavelength of between about 700 nanometers and 1220 nanometers. It is understood that the location of the laser source 86 relative to the light source 20 of the PS-OCT device 14 may be varied. Referring to FIG. 1, the position of the laser source 86 and the direction of the treatment beam 82 may be varied to target the multiple locations of the vitreous opacities 16. For example, a second treatment beam 83 may be produced with the laser source moved to position 87, as shown in FIG. 1. In some embodiments, the laser unit 84 may be movable relative to a center axis A. This may be accomplished through an operator-controlled input device 90, such as a keyboard, mouse, joystick.

In some embodiments, the system 10 incorporates a shared aperture 92 (see FIG. 1) for simultaneous imaging and delivery of treatment to the vitreous opacities 16. The shared aperture 92 may be centered about and perpendicular to the center axis A. The treatment beam 82 may be directed in an off-axis direction, at an off-axis angle 94 between the treatment beam 82 and the center axis A. In some embodiments, the off-axis angle 94 may be at or above 15 degrees. The off-axis angle 94 may be at or above 30 degrees.

The treatment beam 82 may be delivered in various patterns and settings based on the application at hand. It is understood that various modulators or modifiers may be employed by the system 10 to adjust the treatment beam. For example, as shown in FIG. 2, a barrier 96 may be placed close to the eye 12 and adapted to decrease the depth of field for laser delivery. This makes the treatment beam 82 highly convergent. The barrier 96 may be a contact lens worn directly over the cornea 68 of the eye 12.

In summary, the system 10 illustrates a robust way to assist physicians in identifying and treating one or more vitreous opacities 16 in the eye 12. The system 10 enables the generation of tissue-specific contrast images of vitreous floaters in a noninvasive fashion.

The controller C of FIG. 1 includes a computer-readable medium (also referred to as a processor-readable medium), including a non-transitory (e.g., tangible) medium that participates in providing data (e.g., instructions) that may be read by a computer (e.g., by a processor of a computer). Such a medium may take many forms, including, but not limited to, non-volatile media and volatile media. Non-volatile media may include, for example, optical or magnetic disks and other persistent memory. Volatile media may include, for example, dynamic random-access memory (DRAM), which may constitute a main memory. Such instructions may be transmitted by one or more transmission media, including coaxial cables, copper wire and fiber optics, including the wires that comprise a system bus coupled to a processor of a computer. Some forms of computer-readable media include, for example, a floppy disk, a flexible disk, hard disk, magnetic tape, other magnetic medium, a CD-ROM, DVD, other optical medium, a physical medium, a RAM, a PROM, an EPROM, a FLASH-EEPROM, other memory chip or cartridge, or other medium from which a computer can read.

Look-up tables, databases, data repositories or other data stores described herein may include various kinds of mechanisms for storing, accessing, and retrieving various kinds of data, including a hierarchical database, a set of files in a file storage system, an application database in a proprietary format, a relational database management system (RDBMS), etc. Each such data store may be included within a computing device employing a computer operating system such as one of those mentioned above and may be accessed via a network in one or more of a variety of manners. A file system may be accessible from a computer operating system and may include files stored in various formats. An RDBMS may employ the Structured Query Language (SQL) in addition to a language for creating, storing, editing, and executing stored procedures, such as the PL/SQL language mentioned above.

The flowchart shown in the FIGS. illustrates an architecture, functionality, and operation of possible implementations of systems, methods, and computer program products according to various embodiments of the present disclosure. In this regard, each block in the flowchart or block diagrams may represent a module, segment, or portion of code, which comprises one or more executable instructions for implementing the specified logical function(s). It will also be noted that each block of the block diagrams and/or flowchart illustrations, and combinations of blocks in the block diagrams and/or flowchart illustrations, may be implemented by specific purpose hardware-based systems that perform the specified functions or acts, or combinations of specific purpose hardware and computer instructions. These computer program instructions may also be stored in a computer-readable medium that can direct a controller or other programmable data processing apparatus to function in a particular manner, such that the instructions stored in the computer-readable medium produce an article of manufacture including instructions to implement the function/act specified in the flowchart and/or block diagram blocks.

The numerical values of orders (e.g., of quantities or conditions) in this specification, including the appended claims, are to be understood as being modified in each respective instance by the term “about” whether or not “about” actually appears before the numerical value. “About” indicates that the stated numerical value allows some slight imprecision (with some approach to exactness in the value; about or reasonably close to the value; nearly). If the imprecision provided by “about” is not otherwise understood in the art with this ordinary meaning, then “about” as used herein indicates at least variations that may arise from ordinary methods of measuring and using such orders. In addition, disclosure of ranges includes disclosure of each value and further divided ranges within the entire range. Each value within a range and the endpoints of a range are hereby disclosed as separate embodiments.

The detailed description and the drawings or FIGS. are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims. Furthermore, the embodiments shown in the drawings or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

What is claimed is:

1. A system of visualizing a target site in an eye using a polarization sensitive optical coherence tomography (PS-OCT) device, the system comprising:
a controller having at least one processor and at least one non-transitory, tangible memory on which instructions are recorded, the target site being one or more vitreous opacities in a vitreous humor of the eye;
wherein the PS-OCT device includes a source adapted to generate a PS-OCT source beam and a polarizer adapted to control a polarization of the PS-OCT source beam;
wherein the PS-OCT device includes one or more polarization sensitive detectors adapted to detect an interference pattern based in part on a reflected PS-OCT beam, and generate PS-OCT data relating to the interference pattern; and
wherein the controller is configured to receive the PS-OCT data, and determine at least one parameter corresponding to birefringence properties of collagen fibrils in the vitreous humor based on the PS-OCT data, the at least one parameter including a respective spacing of the collagen fibrils; and
wherein the controller is configured to determine a respective location of the one or more vitreous opacities when the at least one parameter is outside a predefined range and generate a control signal adapted for guiding a treatment beam at the respective location of the one or more vitreous opacities.

2. The system of claim 1, wherein the at least one parameter includes a respective orientation of the collagen fibrils.

3. The system of claim 1, wherein the PS-OCT device includes:
a beam splitter adapted to split the PS-OCT source beam into a sample beam propagating in a sample arm and a reference beam propagating in a reference arm, the reference arm having a reference mirror; and
a polarizing beam splitter adapted to split the reflected PS-OCT beam into two orthogonally polarized components, the reflected PS-OCT beam being a combination of respective reflected beams of the sample beam and the reference beam.

4. The system of claim 3, wherein the PS-OCT device includes:
a first quarter-wave plate adapted to convert the sample beam into a polarized sample beam incident upon the target site; and
a second quarter-wave plate adapted to convert the reference beam into a polarized reference beam incident upon the reference mirror.

5. The system of claim 4, wherein:
the two orthogonally polarized components include a vertically polarized component and a horizontally polarized component; and
the one or more polarization sensitive detectors include a vertical detector adapted to receive the vertically polarized component and a horizontal detector adapted to receive the horizontally polarized component.

6. The system of claim 4, wherein the first quarter-wave plate is oriented at an angle of 22.5 degrees and the second quarter-wave plate is oriented at the angle of 45 degrees.

7. The system of claim 1, wherein the PS-OCT device includes a first channel and a second channel adapted to respectively detect a signal from the PS-OCT data in a first orthogonal polarization state and a second orthogonal polarization state, the signal being converted to a fast-Fourier transformed signal.

8. The system of claim 7, further comprising:
a phase retardation mode adapted to display the PS-OCT data, the phase retardation mode being based on a retardation factor (δ), represented as
$$[ δ = {tan}^{- 1} ( F * \frac{{A}_{2}}{{A}_{1}} ) ] ,$$
where F is a calibration factor, and A₁, A₂ are respective amplitudes of the fast-Fourier transformed signal from the first channel and the second channel.

9. The system of claim 7, further comprising:
an optical axis mode adapted to display the PS-OCT data, the optical axis mode being based on an optical factor (θ), represented as
$$[ θ = \frac{π}{2} - \frac{( {ϕ}_{1} - {ϕ}_{2} )}{2} ] ,$$
where ϕ₁ and ϕ2 are respective phases of the fast-Fourier transformed signal from the first channel and the second channel.

10. The system of claim 1, further comprising:
a laser unit adapted to selectively generate the treatment beam directed towards the one or more vitreous opacities, the treatment beam including a plurality of ultra-short laser pulses, the plurality of ultra-short laser pulses defining a respective time duration of between about a femtosecond and about 50 picoseconds.

11. The system of claim 10, wherein the laser unit and the PS-OCT device have a shared aperture for guiding the treatment beam and the PS-OCT beam towards the target site, the shared aperture being centered about a center axis.

12. The system of claim 11, wherein the treatment beam travels at an off-axis angle from the center axis, the off-axis angle being at or above 15 degrees.

13. A method of visualizing a target site in an eye using a polarization sensitive optical coherence tomography (PS-OCT) device in a system having a controller with at least one processor and at least one non-transitory, tangible memory, the method comprising:
generating a PS-OCT source beam, via a light source in the PS-OCT device, the target site being one or more vitreous opacities in a vitreous humor of the eye;
controlling a polarization of the PS-OCT source beam, via a polarizer in the PS-OCT device;
detecting an interference pattern based in part on a reflected PS-OCT beam and generating PS-OCT data relating to the interference pattern, via one or more polarization sensitive detectors in the PS-OCT device;
receiving the PS-OCT data; via the controller;
determining at least one parameter corresponding to birefringence properties of collagen fibrils in the vitreous humor based on the PS-OCT data, via the controller, the at least one parameter including a respective spacing of the collagen fibrils;
determining a respective location of the one or more vitreous opacities when the at least one parameter is outside a predefined range, via the controller; and
generating a control signal adapted for guiding a treatment beam at the respective location of the one or more vitreous opacities, via the controller.

14. The method of claim 13, further comprising:
incorporating a respective orientation of the collagen fibrils in the at least one parameter.

15. The method of claim 13, further comprising:
employing a beam splitter to split the PS-OCT source beam into a sample beam propagating in a sample arm and a reference beam propagating in a reference arm, the reference arm having a reference mirror;
employing a polarizing beam splitter to split the reflected PS-OCT beam into two orthogonally polarized components, the reflected PS-OCT beam being a combination of respective reflected beams of the sample beam and the reference beam;
converting the sample beam into a polarized sample beam incident upon the target site, via a first quarter-wave plate; and
converting the reference beam into a polarized reference beam incident upon the reference mirror, via a first quarter-wave plate.

16. The method of claim 13, further comprising:
orienting the first quarter-wave plate at an angle of 22.5 degrees and orienting the second quarter-wave plate at the angle of 45 degrees.

17. The method of claim 13, further comprising:
selectively generating the treatment beam directed towards the one or more vitreous opacities, via a laser unit, the treatment beam including a plurality of ultra-short laser pulses; and
configuring the laser unit and the PS-OCT device to have a shared aperture for guiding the treatment beam and the PS-OCT beam towards the target site, the shared aperture being centered about a center axis.

18. The method of claim 13, further comprising:
incorporating a first channel and a second channel in the PS-OCT device for respectively detecting a signal from the PS-OCT data in a first orthogonal polarization state and a second orthogonal polarization state, the signal being converted to a fast-Fourier transformed signal.

19. The method of claim 18, further comprising:
displaying the PS-OCT data in a phase retardation mode, the phase retardation mode being based on a retardation factor (δ), represented as
$$[ δ = {tan}^{- 1} ( F * \frac{{A}_{2}}{{A}_{1}} ) ] ,$$
where F is a calibration factor, and A₁, A₂ are respective amplitudes of the fast-Fourier transformed signal from the first channel and the second channel.

20. The method of claim 18, further comprising:
displaying the PS-OCT data in an optical axis mode, the optical axis mode being based on an optical factor (θ), represented as
$$[ θ = \frac{π}{2} - \frac{( {ϕ}_{1} - {ϕ}_{2} )}{2} ] ,$$
where ϕ₁ and ϕ₂ are respective phases of the fast-Fourier transformed signal from the first channel and the second channel.
